(19) United States
(12) Patent Application Publication
(10) Pub. No.: US 2018/0231560 A1
(43) Pub. Date: Aug. 16, 2018

(54) Compositions and Methods for the Capture and Characterization of Circulating Tumor Cells

(71) Applicants: Duke University, Durham, NC (US); JANSSEN DIAGNOSTICS, LLC, Raritan, NJ (US)

(72) Inventors: Galla Chandra Rao, Princeton Junction, NJ (US); Mark C. Connelly, Doylestown, PA (US); Rengasamy BOOMINATHAN, Elkins Park, PA (US); Tian Zhang, Durham, NC (US); Mariano A. Garcia-Blanco, Galveston, TX (US); Andrew J. Armstrong, Chapel Hill, NC (US)

(21) Appl. No.: 15/511,229

(22) PCT Filed: Sep. 15, 2015
(86) PCT No.: PCT/US15/50164

§ 371 Date: Mar. 14, 2017

Related U.S. Application Data
(60) Provisional application No. 62/050,441, filed on Sep. 15, 2014.

(51) Int. Cl. G01N 33/574; C07K 16/30; C07K 16/28

(52) U.S. Cl. CPC G01N 33/57484; G01N 33/57419; G01N 2033/57461; C07K 16/289; C07K 16/2863; C07K 16/30

(57) ABSTRACT

This disclosure provides compositions and methods for the isolation of cells that express c-MET, and in particular circulating tumor cells that express c-MET. The methods can include contacting a biological sample including a c-MET circulating tumor cell with an unbound complex including a capture binding species linked to a solid phase for a time sufficient to allow the unbound complex to bind an extracellular binding domain of the c-MET protein to form a bound complex, and subsequently isolating the bound complex. Compositions, systems, and kits adapted for use with these methods are also provided.

### CROSS-REFERENCE TO RELATED APPLICATIONS

This application is based on, incorporates herein by reference, and claims the benefit of U.S. Provisional Patent Application No. 62/050,441, filed Sep. 15, 2014, and entitled “COMPOSITIONS AND METHODS FOR THE CAPTURE AND CHARACTERIZATION OF CIRCULATING TUMOR CELLS”.

### STATEMENT REGARDING FEDERALLY SPONSORED RESEARCH

This invention was made with government support under Federal Grant No. W81XWH-10-1-0483 awarded by the Department of Defense and Federal Grant No. 5R01-CA127727-03 awarded by the NIH. The government has certain rights in the invention.

### SEQUENCE LISTING

The sequence listing is filed with the application in electronic format only and is incorporated by reference herein. The sequence listing text file “DU4439PCT_ST25.txt” was created on Sep. 15, 2015 and is 35,201 bytes in size.

### BACKGROUND

### 1. Field of the Invention

This invention relates to compositions and methods related to the capture and characterization of circulating tumor cells.

### 2. Description of the Related Art

Predictive biomarkers have the potential to enable personalized approaches to systemic anti-neoplastic therapy. Previous circulating tumor cell (CTC) assays use epithelial cell adhesion molecule (EpCAM) to capture CTCs.

Accordingly, a need exists for methods and systems that can provide improved CTC capture abilities without relying on the standard EpCAM-sensitive techniques.

### SUMMARY

In one aspect of the present disclosure, a method of isolating a c-MET circulating tumor cell from a patient is provided. The method can include one or more of the following steps: obtaining a biological sample from the patient, the biological sample comprising the c-MET CTC; contacting the biological sample or a fraction of the biological sample with an unbound complex, the unbound complex comprising a capture binding species linked to a solid phase, the contacting being for a time sufficient to allow the unbound complex to bind an extracellular binding domain of a c-MET protein on the c-MET CTC to form a bound complex, the capture binding species specifically binding the extracellular binding domain of the c-MET protein; and isolating the bound complex.

In another aspect of the present disclosure, a method of isolating an intact c-MET cell from a patient is provided. The method can include one or more of the following steps: obtaining a biological sample from the patient, the biological sample comprising the intact c-MET cell; contacting the biological sample or a fraction of the biological sample with an unbound complex, the unbound complex comprising a capture binding species linked to a solid phase, the contact being for a time sufficient to allow the unbound complex to bind an extracellular binding domain of a c-MET protein on the intact c-MET cell to form a bound complex, the capture binding species specifically binding the extracellular binding domain of the c-MET protein; isolating the bound complex.

In yet another aspect of the present disclosure, a method of isolating a c-MET circulating tumor cell from a patient is provided. The method can include one or more of the following steps: obtaining a blood sample from the patient, the blood sample comprising a cellular component and a non-cellular component; optionally removing some or all of the non-cellular component from the blood sample; contacting the cellular component with a ferrofluid comprising an unbound complex, the unbound complex comprising a capture binding protein linked to a magnetic particle, the contacting being for a time sufficient to allow the unbound complex to bind an extracellular binding domain of a c-MET protein on the c-MET CTC to form a bound complex, the capture binding species specifically binding the extracellular domain of the c-MET protein; isolating the bound complex from unbound cells of the cellular component; contacting the bound complex with a staining solution; and spectroscopically interrogating the bound complex.

In a further aspect of the present disclosure, a ferrofluid is provided. The ferrofluid can include a magnetic particle linked to a binding species that selectively binds to at least a portion of an extracellular domain of c-MET.

In another aspect of the present disclosure, systems and kits adapted for performing the methods described herein are provided.

These and other features, aspects, and advantages of the present invention will become better understood upon consideration of the following detailed description, drawings and appended claims.

### BRIEF DESCRIPTION OF THE DRAWINGS

FIG. 1 is a flowchart showing a method in accordance with one aspect of the present disclosure.

FIG. 2 is a flowchart showing a method in accordance with one aspect of the present disclosure.

FIG. 3 is a graphical depiction of c-MET CTCs (left) and CD45+ cells (right), where both cells have been DAPI stained in the nucleus, the c-MET CTCs have been captured by an anti-c-MET ferrofluid and stain positive for intracellular c-MET and negative for CD45, and the CD45+ cells have stained positive for CD45.

FIG. 4 is a c-MET immunoblot of cell lines for c-MET expression, as described in Example 1.

FIG. 5 is a plot of enumeration of CTCs captured by c-MET and separated by disease site, as described in Example 1.

FIG. 6 is a plot of enumeration of CTCs captured by EpCAM and separated by disease site, as described in Example 1.

FIG. 7 shows c-MET CTCs isolated from patient A, with 3 and 1 c-MET CTCs in duplicate samples, as described in Example 1.

FIG. 8 shows c-MET CTCs isolated from patient B, with 0 and 4 c-MET CTCs in duplicate samples, as described in Example 1.

FIG. 9 shows c-MET CTCs isolated from patient C, with 52 and 90 c-MET CTCs in duplicate samples, as described in Example 1.

FIG. 10 shows c-MET CTCs isolated from patient D, with 7 and 2 c-MET CTCs in duplicate samples, as described in Example 1.

FIG. 11 is a plot of enumeration of CD45+/CK+ cells captured with c-MET and separated by disease state, as described in Example 1.

FIG. 12 is a boxplot of all samples of CD45+/CK+ cells captured with c-MET from cancer patients versus healthy controls, as described in Example 1.

FIG. 13 shows representative samples of CD45+/CK+ cells captured with c-MET, as described in Example 1.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. In places where ranges of values are given, this disclosure explicitly contemplates other combinations of the lower and upper limits of those ranges that are not explicitly recited. For example, recitation of a value between 1 and 10 or between 2 and 9 also contemplates a value between 1 and 9 or between 2 and 10. Ranges identified as being “between” two values are inclusive of the end-point values. For example, recitation of a value between 1 and 10 includes the values 1 and 10.

Nucleotide sequences described herein and included in the sequence listing represent only the portions of the sequences that code for the corresponding product. For example, the c-MET nucleotide sequence include only the exons that code for the c-MET protein.

Features of this disclosure described with respect to a particular method, apparatus, composition, or other aspect of the disclosure can be combined with, substituted for, integrated into, or in any other way utilized with other methods, apparatuses, compositions, or other aspects of the disclosure, unless explicitly indicated otherwise or necessitated by the context. For clarity, an aspect of the invention described with respect to one method can be utilized in other methods described herein, or in apparatuses or with compositions described herein, unless context clearly dictates otherwise.

### Definitions

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)2 fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C, et al, Nature Biotechnology, 25(11): 1290-1297 (2007) and PCT International Application WO 2001/058956, the contents of each of which are herein incorporated by reference), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody.”

“Antibody fragment” as used herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region. The term “administration” or “administering,” as used herein, refers to providing, contacting, and/or delivery of a cancer treatment by any appropriate route to achieve the desired effect. The cancer treatment may be administered to a subject in numerous ways including, but not limited to, orally, ocularly, nasally, intravenously, topically, as aerosols, suppository, etc. and may be used in combination.

“Binding Protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody, a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Binding species” is used herein to refer to a chemical entity that binds to and forms a complex with a binding partner, such as, for example, a polypeptide, an antigen, a chemical compound or other molecule, or a substrate of any kind. Binding proteins are a subset of binding species.

The term “biomarker” as used herein refers to any quantifiable biological component that is unique to a particular physiological condition (e.g., cancer). A biomarker may be a gene, an mRNA transcribed from said gene, or a protein translated from said mRNA. A measureable increase or decrease, of a biomarker level, relative to a control, such as an individual, group of individuals or populations, or alternatively, relative to subjects with cancer, may provide a diagnosis of a particular physiological condition.

“Cancer” as used herein refers to the uncontrolled and unregulated growth of abnormal cells in the body. Cancerous cells are also called malignant cells. Cancer may invade nearby parts of the body and may also spread to more distant parts of the body through the lymphatic system or bloodstream. Cancers include Adrenocortical Carcinoma, Anal Cancer, Bladder Cancer, Brain Tumor, Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Intraocular Melanoma Eye Cancer, Gallbladder Cancer, Gastric Cancer (Stomach), Extragonadal Germ Cell Tumor, Gestational Trophoblastic Tumor, Head and Neck Cancer, Hypopharyngeal Cancer, Islet Cell Carcinoma, Kidney Cancer (renal cell cancer), Laryngeal Cancer, Acute Lymphoblastic Leukemia, Leukemia, Acute Myeloid, Chronic Lymphocytic Leukemia, Chronic Myelogenous Leukemia, Hairy Cell Leukemia, Lip and Oral Cavity Cancer, Liver Cancer, Non-Small Cell Lung Cancer, Small Cell Lung Cancer, AIDS-Related Lymphoma, Central Nervous System (Primary) Lymphoma, Cutaneous T-Cell Lymphoma, Hodgkin's Disease Lymphoma, Non-Hodgkin's Disease Lymphoma, Malignant Mesothelioma, Melanoma, Merkel Cell Carcinoma, Metasatic Squamous Neck Cancer with Occult Primary, Multiple Myeloma and Other Plasma Cell Neoplasms, Mycosis Fungoides, Myelodysplasia; Syndrome, Myeloproliferative Disorders, Nasopharyngeal Cancer, Neuroblastoma, Oral Cancer, Oropharyngeal Cancer, Osteosarcoma, Ovarian Epithelial Cancer, Ovarian Germ Cell Tumor, Pancreatic Cancer (Exocrine), Pancreatic Cancer (Islet Cell Carcinoma), Paranasal Sinus and Nasal Cavity Cancer, Parathyroid Cancer, Penile Cancer, Pituitary Cancer, Plasma Cell Neoplasm, Prostate Cancer, Rhabdomyosarcoma, Rectal Cancer, Renal Cell Cancer (cancer of the kidney), Transitional Cell Renal Pelvis and Ureter, Salivary Gland Cancer, Sezary Syndrome, Skin Cancer, Small Intestine Cancer, Soft Tissue Sarcoma, Testicular Cancer, Malignant Thymoma, Thyroid Cancer, Urethral Cancer, Uterine Cancer, Unusual Cancer of Childhood, Vaginal Cancer, Vulvar Cancer, and Wilms' Tumor.

“Circulating tumor cells”, “CTC” and “CTCs” as used interchangeably herein refers to cells that have shed into the vasculature from a primary tumor and circulate in the bloodstream. CTCs are considered seeds for subsequent growth of additional tumors (metastasis) in vital distant organs, triggering a mechanism that is responsible for the vast majority of cancer-related deaths.

“c-MET cell” as used herein refers to a cell that expresses c-MET. “c-MET CTC” as used herein refers to a circulating tumor cell that expresses c-MET and does not express CD45.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum or plasma sample, in accordance with the methods described herein and other methods known in the art. Some components can be in solution or lyophilized for reconstitution for use in an assay.

The term “effective dosage” as used herein means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as ³H, ¹⁴C, ³²P, ³³P, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3 ′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entirety). An acridinium compound can be used as a detectable label in a homogeneous chemiluminescent assay (see, e.g., Adamczyk et al, Bioorg. Med. Chem. Lett. 16: 1324-1328 (2006); Adamczyk et al, Bioorg. Med. Chem. Lett. 4: 2313-2317 (2004); Adamczyk et al, Biorg. Med. Chem. Lett. 14: 3917-3921 (2004); and Adamczyk et al, Org. Lett. 5: 3779-3782 (2003)).

The term “link” or “linked” as used herein refers to direct or indirect chemical linking of two species.

The term “normal subject” as used herein means a healthy subject, i.e. a subject having no clinical signs or symptoms of cancer. The normal subject is clinically evaluated for otherwise undetected signs or symptoms of cancer, which evaluation may include routine physical examination and/or laboratory testing.

The term “predetermined cutoff and “predetermined level” as used herein means an assay cutoff value that is used to assess diagnostic, prognostic, or therapeutic efficacy results by comparing the assay results against the predetermined cutoff/level, where the predetermined cutoff/level already has been linked or associated with various clinical parameters (e.g., presence of disease, stage of disease, severity of disease, progression, non-progression, or improvement of disease, etc.). The disclosure provides exemplary predetermined levels. However, it is well-known that cutoff values may vary depending on the nature of the immunoassay (e.g., antibodies employed, reaction conditions, sample purity, etc.). It further is well within the ordinary skill of one in the art to adapt the disclosure herein for other immunoassays to obtain immunoassay-specific cutoff values for those other immunoassays based on the description provided by this disclosure. Whereas the precise value of the predetermined cutoff/level may vary between assays, the correlations as described herein should be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. A pretreatment reagent may be homogeneous (not requiring a separation step) or heterogeneous (requiring a separation step). With use of a heterogeneous pretreatment reagent, there is removal of any precipitated analyte binding proteins from the test sample prior to proceeding to the next step of the assay. The pretreatment reagent optionally can comprise: (a) one or more solvents and salt, (b) one or more solvents, salt and detergent, (c) detergent, (d) detergent and salt, or (e) any reagent or combination of reagents appropriate for cell lysis and/or solubilization of analyte.

“Prostate cancer” as used herein refers to a type of cancer that develops in the prostate. Prostate cancer may be slow growing or aggressive, in which the cancer cells metastasize from the prostate to other parts of the body, particularly the bones and lymph nodes. “Metastatic prostate cancer” refers to prostate cancer that spreads outside the prostate gland to the lymph nodes, bones, or other areas. “Castration resistant prostate cancer” refers to prostate cancer disease progression despite androgen-deprivation therapy, which may present as one or any combination of a continuous rise in serum levels of prostate-specific antigen, progression of pre-existing disease, or appearance of new metastases.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction to comprise a “sensitivity panel.”

The term “reference activity level” or “reference” as used herein means an activity level of the biomarker in a sample group that serves as a reference against which to assess the activity level in an individual or sample group.

The term “risk assessment,” “risk classification,” “risk identification,” or “risk stratification” as used herein interchangeably, means an evaluation of factors including biomarkers, to predict the risk of occurrence of future events including disease onset or disease progression, so that treatment decisions regarding the subject may be made on a more informed basis.

The term “sample,” “test sample,” “specimen,” “biological sample,” “sample from a subject,” or “subject sample” as used herein interchangeably, means a sample or isolate of blood, tissue, urine, serum, plasma, amniotic fluid, cerebrospinal fluid, placental cells or tissue, endothelial cells, leukocytes, or monocytes, can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

The term also means any biological material being tested for and/or suspected of containing an analyte of interest. The sample may be any tissue sample taken or derived from the subject. In some embodiments, the sample from the subject may comprise protein. Any cell type, tissue, or bodily fluid may be utilized to obtain a sample. Such cell types, tissues, and fluid may include sections of tissues such as biopsy and autopsy samples, frozen sections taken for histological purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

Methods well-known in the art for collecting, handling and processing urine, blood, serum and plasma, and other body fluids, are used in the practice of the present disclosure. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples, a pre-processed archived sample, etc.), pretreatment of the sample is an option that can be performed for mere convenience (e.g., as part of a protocol on a commercial platform). The sample may be used directly as obtained from the subject or following pretreatment to modify a characteristic of the sample. Pretreatment may include extraction, concentration, inactivation of interfering components, and/or the addition of reagents.

“Solid phase” refers to any material that is insoluble, or can be made insoluble by a subsequent reaction. The solid phase can be chosen for its intrinsic ability to attract and immobilize a capture agent. Alternatively, the solid phase can have affixed thereto a linking agent that has the ability to attract and immobilize the capture agent. For example, the linking agent can include a charged substance that is oppositely charged with respect to the capture agent itself or to a charged substance conjugated to the capture agent. In general, the linking agent can be any binding partner (preferably specific) that is immobilized on (attached to) the solid phase and that has the ability to immobilize the capture agent through a binding reaction. The linking agent enables the indirect binding of the capture agent to a solid phase material before the performance of the assay or during the performance of the assay. For examples, the solid phase can be plastic, derivatized plastic, magnetic, paramagnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject”, “patient” or “subject in the method” as used herein interchangeably, means any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human. In some embodiments, the subject or subject may be a human or a non-human. In some embodiments, the subject may be a human subject at risk for developing or already having cancer.

“Treat”, “treating” or “treatment” are each used interchangeably herein to describe reversing, alleviating, or inhibiting the progress of a disease, such as cancer, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an antibody or pharmaceutical composition of the present invention to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

This disclosure provides for the detection, identification, isolation, capture, enrichment, or enumeration of cells that amplify expression of the MET oncogene. The cells can be CTCs. CTCs with MET amplification can be detected in patients with metastatic, treatment refractory gastrointestinal (GI) and genitourinary (GU) malignancies. c-MET is an expression product of the MET oncogene. Isolation of c-MET CTCs in real-time can improve understanding of the timing of MET amplification in patients and can help facilitate selective study of c-MET inhibitors in patients.

Methods of the Present Disclosure

This disclosure provides methods of detecting, identifying, isolating, capturing, enriching, or enumerating c-MET cells, and in particular c-MET CTCs, from a biological sample of a patient or from a patient.

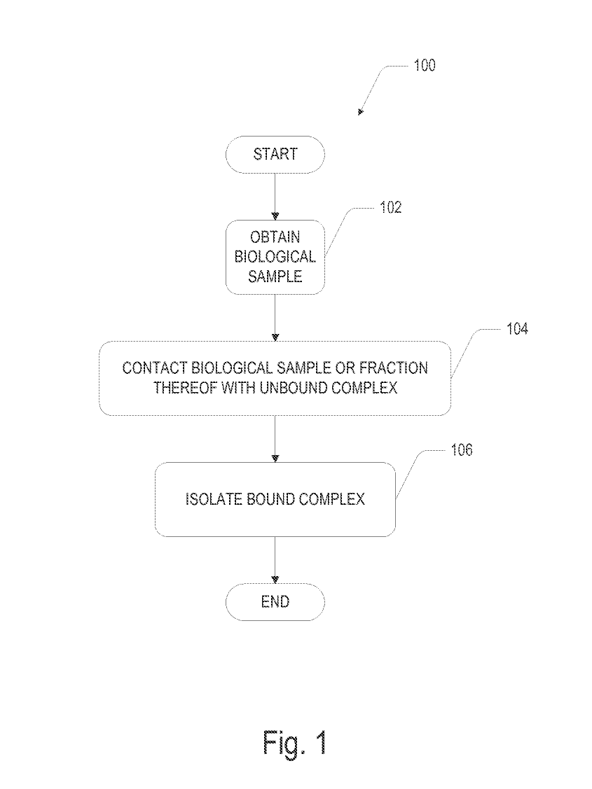

Referring to FIG. 1, a method 100 of isolating a c-MET circulating tumor cell or an intact c-MET cell from a patient is disclosed. At process block 102, the method 100 can include obtaining a biological sample from a patient. At process block 104, the method 100 can include contacting the biological sample or a fraction of the biological sample with an unbound complex for a time sufficient to allow the unbound complex to bind an extracellular domain of a c-MET protein. At process block 106, the method 100 can include isolating the bound complex.

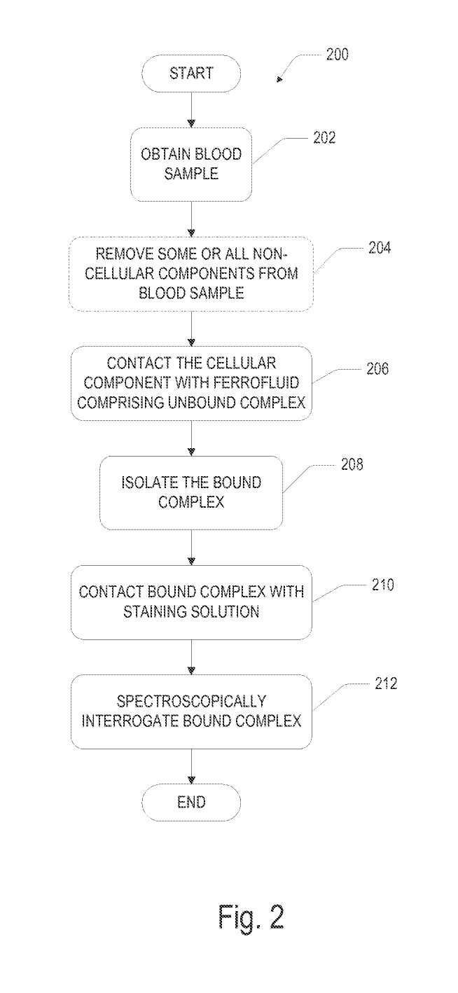

Referring to FIG. 2, a method 200 of isolating a c-MET circulating tumor cell from a patient is disclosed. At process block 202, the method 200 can include obtaining a blood sample from the patient, the blood sample comprising a cellular component and a non-cellular component. At process block 204, the method 200 can optionally include removing some or all of the non-cellular component from the blood sample. At process block 206, the method 200 can include contacting the cellular component with a ferrofluid comprising an unbound complex. At process block 208, the method 200 can include isolating the bound complex from unbound cells of the cellular component. At process block 210, the method 200 can include contacting the bound complex with a staining solution. At process block 212, the method 200 can include spectroscopically interrogating the bound complex.

In certain aspects, the biological sample can comprise a c-MET CTC. In certain aspects, the biological sample can comprise an intact c-MET cell. In certain aspects, the biological sample can be a blood sample.

In certain aspects, the methods can include removing at least a portion of the biological sample that does not include the c-MET CTC or the c-MET cell. The removing can be by way of aspiration. In certain aspects, the methods can comprise aspirating a plasma portion of a blood sample.

In certain aspects, the unbound complex can comprise a capture binding species linked to a solid phase. The capture binding species can specifically bind the extracellular binding domain of the c-MET protein. The capture binding species can be a capture binding protein. The capture binding species can be an anti-c-MET antibody.

The c-MET protein can have a polypeptide sequence of SEQ ID NO: 1. The c-MET protein can be identified by GenBank accession number M35073.

The extracellular binding domain of the c-MET protein can have a polypeptide sequence of residues 25 to 932 of SEQ ID NO: 1. In certain aspects, the capture binding species can specifically bind a protein of interest having a sequence that is at least 90%, at least 95%, at least 99%, or at least 99.9% homologous to the polypeptide sequence of residues 25 to 932 of SEQ ID NO: 1.

The capture binding protein can include an extracellular binding domain of the c-MET protein binding portion selected from the group consisting of HGF R/c-MET Antibody clone 95106 (available commercially from Novus® Biologicals, Littleton, Colo.), HGF R/c-MET Antibody clone EP1454Y (available commercially from Novus® Biologicals, Littleton, Colo.), and HGF R/c-MET Antibody clone L6E7 (available commercially from Cell Signaling Technologies, Beverly, Mass.). The extracellular binding domain of the c-MET protein binding portion can have a structure that is at least 90%, at least 95%, at least 99%, or at least 99.9% homologous to the structure of HGF R/c-MET Antibody clone 95106 (available commercially from Novus® Biologicals, Littleton, Colo.), HGF R/c-MET Antibody clone EP1454Y (available commercially from Novus® Biologicals, Littleton, Colo.), and HGF R/c-MET Antibody clone L6E7 (available commercially from Cell Signaling Technologies, Beverly, Mass.).

The solid phase can be a magnetic particle, as described herein.

Isolated bound complexes can be contacted by a staining solution, in order to impart various selective stains to a c-MET CTC or intact c-MET cell. The staining solution can include one or more staining complexes or staining species. The staining complex can include a detectable label linked to a staining binding species. The detectable label can be any label that is detectable by known interrogation methods.

In order to confirm the expression of c-MET, the methods described herein can include intracellular staining of the cells to map the intracellular expression of c-MET. The staining binding species can be an anti-c-MET, and in particular, the staining binding species can specifically bind the intracellular binding domain of the c-MET protein. The intracellular binding domain of the c-MET protein can have a polypeptide sequence of residues 956 to 1390 of SEQ ID NO: 1. In certain aspects, the staining binding species can specifically bind a protein of interest having a sequence that is at least 90%, at least 95%, at least 99%, or at least 99.9% homologous to the polypeptide sequence of residues 956 to 1390 of SEQ ID NO: 1.

The staining binding protein can include an intracellular binding domain of the c-MET protein binding portion selected from the group consisting of HGF R/c-MET Antibody clone E999 (available commercially from Novus® Biologicals, Littleton, Colo.) and HGF R/c-MET Antibody clone 3D4 (available commercially from Thermo Fisher Scientific, Grand Island, N.Y.). The intracellular binding domain of the c-MET protein binding portion can have a structure that is at least 90%, at least 95%, at least 99%, or at least 99.9% homologous to the structure of HGF R/c-MET Antibody clone E999 (available commercially from Novus® Biologicals, Littleton, Colo.) and HGF R/c-MET Antibody clone 3D4 (available commercially from Thermo Fisher Scientific, Grand Island, N.Y.).

In addition to investigating the intracellular expression of c-MET, the methods described herein can utilize staining for several other purposes, including but not limited to, excluding leukocytes, identifying cells having intact nuclei, and other staining protocols known to those having ordinary skill in the art.

Given that CTCs are extraordinarily rare relative to other circulating cells, the isolation of CTCs can involve the identification and exclusion of cells expressing the pan-leukocyte marker CD45. Circulating CD45 negative cells are not necessarily tumor-derived, however, but instead may represent normal blood vessel or stromal cells, circulating mesenchymal cells or stem cells, or other host cells that exist in rare quantities in the circulation. Circulating endothelial cells result from blood vessel wall turnover, and bone marrow-derived endothelial progenitor cells may circulate in the setting of neovascularization of ischemic tissue and tumor formation. These cells are all CD45 negative. Also CD45 negative, mesenchymal stromal cells (MSCs) are a more diverse group of cells that may be bone marrow-, peripheral blood-, or fat-derived. MSCs are multipotent cells that may differentiate into a variety of stromal cell types, circulate in inflammatory disorders, and are under active investigation for use in regenerative medicine and other conditions. The significance of circulating MSCs in cancer remains unclear. Thus, CTC detection methods can involve distinguishing tumor cells from a range of other rare non-tumor cells in the circulation. Confirmation of CTCs may include staining with DAPI. Confirmation of CTCs may include identification and inclusion of cells expressing a cytokeratin. For example, a CTC may be confirmed if DAPI staining is positive, cytokeratin expression is positive, and CD45 expression is negative. The detection of CD45 or cytokeratins can be performed using antibodies against CD45 or cytokeratin, wherein the antibodies are labeled.

Cluster of differentiation 45 (CD45), also known as protein tyrosine phosphatase, receptor type, C and leukocyte common antigen, is encoded by the PTPRC gene. CD45 is used to identify leukocytes. CD45 can have a polypeptide sequence of SEQ ID NO: 2 or a polypeptide sequence of SEQ ID NO: 2 with a deletion of residues 32 to 192. CD45 can be identified by GenBank accession number Y00062.

An antibody that binds to CD45 may be used to detect CD45. An antibody that binds to CD45 can be selected from the group consisting of CD45 Antibody clones HI30 (available commercially from eBioscience, San Diego, Calif.), 2D1 (available commercially from eBioscience, San Diego, Calif.), 2B11 (available commercially from Novus® Biologicals, Littleton, Colo.), MEM-28 (available commercially from Novus® Biologicals, Littleton, Colo.), SPM570 (available commercially from Novus® Biologicals, Littleton, Colo.), and F10-89-4 (available commercially from Genway Biotech Inc., San Diego, Calif.).

Cytokeratins are keratin-containing intermediate filaments found in the intracytoplasmic cytoskeleton of epithelial tissue. Cytokeratin-expressing cancer cells lose their cytokeratin expression after undergoing epithelial-mesenchymal transition, with up to 20% of cells having no detectable cytokeratin. A protein other than cytokeratin may identify a pure mesenchymal CTC. In certain aspects, the methods described herein can detect the expression of cytokeratins 8, 18, or 19. Cytokeratin 8 can have a polypeptide sequence of SEQ ID NO: 3. Cytokeratin 8 can be identified by GenBank accession number BC000654. Cytokeratin 18 can have a polypeptide sequence of SEQ ID NO: 4. Cytokeratin 18 can be identified by NCBI accession number NM_199187. Cytokeratin 19 can have a polypeptide sequence of SEQ ID NO: 5. Cytokeratin 19 can be identified by NCBI accession number NM_002276.

An antibody that binds to cytokeratin 8, 18, 19, or a combination thereof may be used to detect cytokeratin 8, 18, 19, or a combination thereof. An antibody that binds to cytokeratin 8, 18, 19, or a combination thereof can be selected from the group consisting of cytokeratin Antibody clones CK3-6H5 (available commercially from Miltenyi Biotec Inc., San Diego, Calif.), TS1-DC10-BA17 (available commercially from antibodies-online Inc., Atlanta, Ga.), and 2A4 (available commercially from Abcam® plc, Cambridge, Mass.).

DAPI, also known as 4′,6-diamidino-2-phenylindole, is a fluorescent stain that binds strongly to A-T rich regions in DNA. It is used extensively in fluorescence microscopy. DAPI can pass through an intact cell membrane therefore it can be used to stain both live and fixed cells.

Spectroscopically interrogating bound complexes can include spectroscopic and microscopic methods known to those having ordinary skill in the art to be useful for the detection of labels, as described herein. Examples of suitable spectroscopic interrogation methods include, but are not limited to, fluorescence in situ hybridization (FISH), fluorescence microscopy, fluorescence spectroscopy, scintillation detection methods, and the like.

This disclosure also provides methods of detecting cancer, treating cancer, monitoring progression of cancer, or determining a cancer prognosis for a patient. This disclosure also provides methods for predicting responsiveness to a course of treatment for a patient having cancer.

Aspects also relate to methods of predicting responsiveness of a subject to a cancer drug. The methods may comprise determining the level of expression of c-MET in a sample from the subject. The level of expression of c-MET may be used to obtain a gene expression pattern in CTCs for the subject. The methods may further comprise predicting responsiveness of the subject to the cancer drug based on the gene expression pattern obtained. Genome variation in CTCs from the subject may also be determined.

Also provided are methods of providing a cancer prognosis to a subject. The methods may comprise determining the level of expression of c-MET in a sample from the subject. The level of expression of c-MET may be used to determine the number of CTCs in the sample. The CTCs may be captured using the extracellular binding domain of c-MET. The level of expression of c-MET may be used to determine a gene expression pattern in the CTCs for the subject. A prognosis may be provided to the subject based on the gene expression pattern obtained.

Also provided are methods for following the progress of cancer in a subject. The methods may comprise determining the level of expression of c-MET in samples from the subject at a first and a second time, and comparing the first and second levels of expression. The level of expression of c-MET in the sample may be determined over time, such as following initiation of a new cancer therapy. The level of expression of c-MET in the sample may be used to determine the number or amount of CTCs. An increase between the first and second levels may indicate progression of the cancer. A decrease between the first and second levels may indicate remission or response of the cancer to the therapy. No difference between the first and second levels may indicate arrest or stability in the progression of the cancer.

Also provided are methods of screening for cancer in a subject. The methods may comprise determining the level of expression of c-MET in a sample from the subject. The level of expression of c-MET may be used to determine the amount or number of CTCs in the subject. The level of expression of c-MET may be compared to a normal or control sample. An increased level of c-MET may indicate presence of cancer in the subject.

In certain aspects, the patient can have cancer. In certain aspects, the cancer can be gastrointestinal cancer or genitourinary cancer. The cancer can be gastric cancer, pancreatic cancer, renal cancer, colorectal cancer, bladder cancer, or prostate cancer. In certain aspects, the cancer can be gastric cancer, colorectal cancer, or renal cell carcinoma.

Compositions of Matter of the Present Disclosure

This disclosure provides a ferrofluid that is suitable for use in the methods, systems, and kits described herein, as could be identified by a person having ordinary skill in the art.

The ferrofluid can include a carrier component and a suspended particle component.

The carrier component can include any liquid that has suitable physical properties, including suitable viscosity, chemical inertness, magnetic inertness, and the like.

The suspended particle component can include an unbound complex. The unbound complex can include a magnetic particle linked to a capture binding species that selectively binds to at least a portion of the extracellular domain of c-MET.

The capture binding species can have the properties described elsewhere herein.

The magnetic particle can be any nanoparticle that is suitable for use in the methods, systems, and kits described herein, as could be identified by a person having ordinary skill in the art.

In certain aspects, the magnetic particle can consist of a homogenous magnetic material. In certain aspects, the magnetic particle can include a magnetic core, a non-magnetic coating surrounding the magnetic core, and a binding species connected to the magnetic core or the coating. In certain aspects, the magnetic particle can include a non-magnetic core, a magnetic coating on at least part of the non-magnetic core, and a binding species connected to the non-magnetic core or the magnetic coating.

In certain aspects, the magnetic particle can be a magnetic microparticle or a magnetic nanoparticle.

In certain aspects, the magnetic particles, magnetic cores, magnetic coatings, or magnetic materials described herein can be ferromagnetic or ferromagnetic.

In certain aspects, the ferromagnetic or ferrimagnetic particle, the ferromagnetic or ferrimagnetic core, or the ferromagnetic or ferrimagnetic coating can comprise a ferromagnetic or ferrimagnetic material selected from the group consisting of Fe, Fe₃O₄, Fe₂O₃, CuOFe₂O₃, Co, CrO₂, Dy, EuO, (Ga,Mn)As, Gd, MgOFe₂O₃, MnAs, MnBi, MnSb, MnOFe₂O₃, Ni, NiOFe₂O₃, SmCo, Y₃Fe₅O₁₂, and alloys and combinations thereof. Examples of suitable ferromagnetic or ferromagnetic alloys include, but are not limited to, alnico, bismanol, cubic ferrites, fernico, hexagonal ferrites, metglas MKM steel, permalloy, pyrrhotite, suessite, yttrium iron garnet, and the like.

In certain aspects, the non-ferromagnetic particle or the non-ferromagnetic coating can comprise a non-ferromagnetic and/or non-ferrimagnetic material selected from the group consisting of silica, styrene, combinations thereof, and the like.

Systems of the Present Disclosure

Systems of the present disclosure can include one or more of the following: an aspirator for removing plasma and/or other ancillary components from biological samples; a fluid distributor for adding ferrofluids and/or buffers to samples; an incubation chamber capable of incubating the biological samples with ferrofluids at a desired temperatures; an aspirator for removing unbound cells from the ferrofluid-contacted biological sample; a manipulable magnet for removing magnetic complexes; a fluid distributor for adding staining reagents to the isolated magnetic complexes; a fluorescence spectroscopy or microscopy instrument.

The system can include any aspirator known to those having ordinary skill in the art to be suitable for removing plasma and other ancillary components from biological samples, such as blood, while leaving the cellular components of the biological sample intact. The system can also include an aspirator known to those having ordinary skill in the art to be suitable for removing unbound cells from a sample that includes bound complexes. Examples of suitable aspirators include, but are not limited to, the aspirator that is included in the CELLTRACKS® AUTOPREP® system (available commercially from Janssen Diagnostics, LLC), and the like.

The system can include a fluid distributor known to those having ordinary skill in the art to be suitable for adding ferrofluids, buffers, and/or staining reagents to various samples. Examples of suitable fluid distributors include, but are not limited to, the fluid distributors that are included in the CELLTRACKS® AUTOPREP® system (available commercially from Janssen Diagnostics, LLC), and the like.

The system can include an incubation chamber known to those having ordinary skill in the art to be suitable for incubating samples, such as those described herein. Examples of suitable incubation chambers include, but are not limited to, the incubation chambers that are included in the CELLTRACKS® AUTOPREP® system (available commercially from Janssen Diagnostics, LLC), and the like.

The system can include a manipulable magnet known to those having ordinary skill in the art to be suitable for removing or isolating bound magnetic complexes. Examples of suitable manipulable magnets include, but are not limited to, the manipulable magnets that are included in the CELLTRACKS® AUTOPREP® system (available commercially from Janssen Diagnostics, LLC), and the like.

The system can include a fluorescence spectroscopy or microscopy instrument known to those having ordinary skill in the art to be suitable for inquisition of the labels described herein. Examples of suitable fluorescent spectroscopy or microscopy instruments include, but are not limited to, the fluorescent spectroscopy or microscopy instruments that are included in the CELLTRACKS® ANALYZER II® system (available commercially from Janssen Diagnostics, LLC), and the like.

Kits of the Present Disclosure

Kits of the present disclosure can include one or more of the following: all or part of the compositions of matter described herein; all or part of the systems described herein; instructions for executing the methods described herein; and instructions for interpreting data acquired using the compositions of matter and systems described herein.

### EXAMPLES

### Example 1. c-MET CTC Assay

The CellSearch® assay for the traditional EpCAM CTC capture was used, as previously described in Shaffer D R, Leversha M A, Danila D C, et al. Circulating tumor cell analysis in patients with progressive castration-resistant prostate cancer. Clinical cancer research: an official journal of the American Association for Cancer Research 2007; 13:2023-9, which is incorporated herein in its entirety by reference. For novel c-MET CTC capture, an anti-c-MET ferrofluid was designed for the immunomagnetic capture of CTCs. An antibody targeting extracellular c-MET (clone L6E7, Cell Signaling Technologies, Beverly, Mass.) was used in conjugation with iron nanoparticles via a biotin-streptavidin interaction, similar to the CellSearch® method, as previously described in Allard W J, Matera J, Miller M C, et al. Tumor cells circulate in the peripheral blood of all major carcinomas but not in healthy subjects or patients with nonmalignant diseases. Clinical cancer research: an official journal of the American Association for Cancer Research 2004; 10:6897-904, which is incorporated herein in its entirety by reference. After capture and enhancement, fluorescent reagents were added for identification and enumeration of the target cells. These reagents included a confirmatory antibody targeting intracellular c-MET (clone 3D4, Invitrogen, Carlsbad, Calif.) conjugated to phycoerythrin (PE), 4′,6-diamidino-2-phenylindole (DAPI), an anti-CD45 monoclonal antibody (Veridex clone HI30) conjugated to allophycocyanin (APC), and antibodies directed to cytokeratins 8, 18, and 19 conjugated to fluorescein isothiocyanate (FITC). The processed reagent/sample mixture is dispensed by the CellTracks® AutoPrep System into a cartridge that is inserted into a MagNest® device and processed on the CellTracks® Analyzer II. Circulating tumor cells were defined as c-MET positive and DAPI positive nucleated and intact cells lacking CD45, without using cell size in the definition.

Cell lines were obtained from ATCC and grown to confluence in either Dulbecco's Modified Eagle Medium (DMEM) or Roswell Park Memorial Institute (RPMI) medium and harvested in phosphate buffer saline (PBS). The following cell lines were used for assay characterization: SNU5 cells (MET amplified, EpCAM positive gastric cancer cell line), A549 (c-MET expressing, EpCAM positive lung cancer cell line), SiHA (c-MET expressing, EpCAM positive cervical cancer cell line), PC3 (c-MET expressing, EpCAM positive prostate cancer cell line), HeLa cells (c-MET expressing, EpCAM negative cervical cancer cell line), BT549 (c-MET expressing, EpCAM negative breast adenocarcinoma cell line), and LnCAP (c-MET negative, EpCAM positive prostate cancer cell line) were used for assay characterization. The assay was tested for sensitivity and specificity using these cell lines spiked in buffer, as well as spiked into whole blood from healthy volunteers recruited at Duke University Medical Center through an IRB approved protocol and after informed consent. Cells were counted and diluted, and between 30 and 10,000 cells were spiked in each sample tested.

Cells were harvested with cell dissociation buffer and fixed with 1% PFA. Cells were washed with PBS and then permeabilized with 0.1% triton in PBS for 30 minutes at room temperature, then blocked with 10% goat-serum in PBS. For staining the cell density was adjusted to 1×10⁶ cells/mL. Two samples were made for each cell line. The first sample was stained with c-MET (external, CellSignaling 8741 with goat-anti-mouse IgG-488, A11001), and the second sample was stained with c-MET (internal, LifeTechnologies, 37-0100 antibody-labeled with Z25102-A488) and EpCAM (from Veridex mouse-antibody labeled with Z25005-A647). After incubating and staining, the cells were washed with PBS and then divided equally for either flow cytometry (BD Canto II) or staining with DAPI. Final analysis was performed with fluorescence microscopy.

All patients (“pts”) were enrolled at the Duke Cancer Institute in Durham, N.C. We designed a prospective feasibility study to capture c-MET expressing CTCs from patients with gastrointestinal (including gastroesophageal, pancreatic, and colorectal adenocarcinomas) and genitourinary (including prostate, renal cell carcinoma, and bladder urothelial carcinoma) malignancies. All patients were older than age 18, had histologically confirmed malignancies as well as clinical or radiographic evidence of metastatic disease, and were enrolled before the initiation of a new systemic therapy. All patients had progression of disease on or following their most recent systemic therapy, with disease progression defined as radiographic progression or clinical progression of disease including cutaneous or palpable lesions, as well as new fluid accumulation such as pleural effusions or ascites. Patients with metastatic castration resistant prostate cancer could have disease progression defined as 2 consecutive PSA levels greater than the PSA nadir achieved on androgen deprivation therapy and their most recent therapy. Due to the aggressive nature of pancreatic cancers as well as non-clear cell renal cell carcinomas, these patients could be enrolled prior to their first systemic therapy. For clear cell renal cell carcinomas, patients were eligible if they had disease progression within a year of starting a VEGF targeting therapy.

All study subjects signed informed consent to participate in this Duke IRB approved, single institution, investigator initiated study. A single peripheral blood draw was performed. Subjects had peripheral whole blood collected into four 10 mL Cellsave® vacutainers. Samples were stored at ambient room temperature and processed within 48 hours of collection. 7.5 mL of whole blood was run per sample, and duplicate samples were processed for c-MET and EpCAM capture.

CTC enumeration for c-MET and EpCAM capture was performed as described above. When c-MET CTCs were present, DNA fluorescent in situ hybridization (FISH) was performed using a dual color FISH probe for c-MET and SE7. A repeat free FISH probe for c-MET was prepared using BAC clones RP11-11406 and CTD-2369N14 and labeled with PlatinumBright 550 (Leica Biosystems) as previously described in Swennenhuis J F, Foulk B, Coumans F A, Terstappen L W. Construction of repeat-free fluorescence in situ hybridization probes. Nucleic acids research 2012; 40:e20, which is incorporated herein in its entirety by reference. The chromosome 7 centromere probe (SE7) was labeled with Platinum Bright 415 and was obtained from Leica Biosystems. Methods for performing FISH on CTC have been previously described in Swennenhuis J F, Tibbe A G, Levink R, Sipkema R C, Terstappen L W. Characterization of circulating tumor cells by fluorescence in situ hybridization. Cytometry Part A: the journal of the International Society for Analytical Cytology 2009; 75:520-7, which is incorporated herein its entirety by reference.

Descriptive statistics were used to describe clinical parameters and CTC enumeration for c-MET and EpCAM capture. Prevalence of detectable CTCs using c-MET capture was calculated as the proportion of patients with at least one c-MET CTC that was validated by replicate. The Wilcoxon rank-sum test was used to assess the difference in the number of c-MET+/CD45+/CK+ cells among cancer patients and healthy controls.

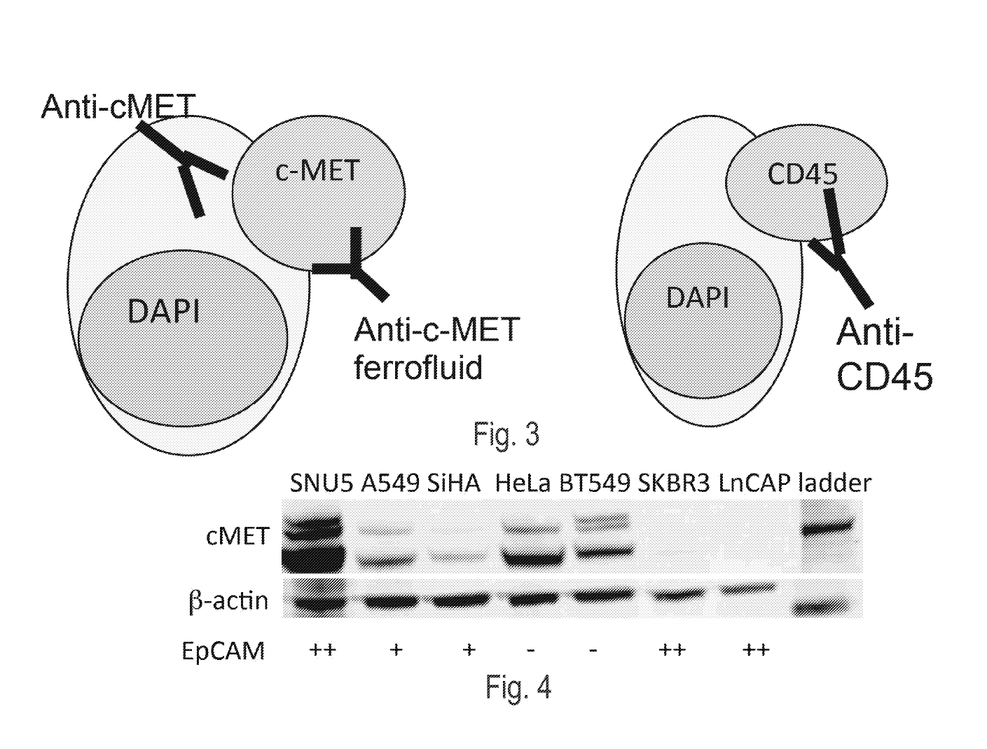

c-MET CTCs were defined as nucleated intact cells captured using a ferromagnetic antibody directed against the c-MET extracellular domain, positive for c-MET (intracellular domain) and DAPI, negative for the leukocyte marker CD45, and without any specific size criteria (FIG. 3). As proof of the principal experiment to show that c-MET CTCs can be captured, cell lines were first characterized for c-MET by immunoblot (FIG. 4). The MET amplified SNU5 cell line was used as a positive control, and the c-MET negative LnCAP cell line and donor leukocytes were used as negative controls, with cells in buffer or spiked into peripheral blood samples from healthy controls. Recovery of c-MET cells with the c-MET CTC assay ranged from 20-40% in c-MET expressing cell lines to 60-80% for the MET amplified SNU5 and c-MET overexpressing HeLa cell lines (see, Table 1—MET amplified cell line: SNU5; c-MET high-expressing, EpCAM negative cell lines: BT549, HeLa; c-MET high-expressing, EpCAM positive cell lines: PC3, AT549; c-MET low-expressing cell line: SiHA; c-MET negative: LnCAP). None of the spiked LnCAP cells were captured with the c-MET CTC assay, either in buffer or in healthy control blood; therefore the specificity of the c-MET CTC assay was 100%.

TABLE 1

| | | % cells | | % cells | |
|---|---|---|---|---|---|
| | | captured | | capture | |
| | | c-MET | | EpCAM | |
| | | assay mean | | assay mean | |
| Cell Line | c-MET/EpCAM Status | (std dev) | | (std dev) | |
| SNU5 | MET amplified | 65% | (15) | 70% | (21) |
| A549 | c-MET high-expressing, | 32% | (17) | 51% | (N/A) |
| | EpCAM positive | | | | |
| SiHA | c-MET low-expressing, | 18% | (8) | 23% | (N/A) |
| | EpCAM positive | | | | |
| HeLa | c-MET high-expressing, | 66% | (15) | 0% | (0) |
| | EpCAM negative | | | | |
| BT549 | c-MET high-expressing, | 35% | (N/A) | 0% | (0) |
| | EpCAM negative | | | | |
| PC3 | c-MET high-expressing, | 40% | (2) | 88% | (N/A) |
| | EpCAM positive | | | | |
| LnCAP | c-MET negative | 0% | (0) | N/A | |
| Healthy control | n/a | 0% | (0) | 0% | (0) |

The efficiency of c-MET capture to the current EpCAM-based capture methods were then compared. Most of the cell lines were captured with both c-MET capture as well as the EpCAM capture assay. However, in order to determine if the c-MET assay can identify CTC that have lost their epithelial phenotype and EpCAM expression, HeLa and BT549 cell lines were tested, which are known to express c-MET and lack EpCAM. These cells were captured by the c-MET assay with a sensitivity of about 65% and 34.6%, respectively, but not captured by the EpCAM assay as expected. SNUS, HeLa, and SIHA cell lines were also characterized for EpCAM and c-MET expression by immunofluorescence confirming c-MET expression and lack of EpCAM.

We next evaluated the c-MET CTC assay in a range of patients with metastatic gastrointestinal and genitourinary malignancies. Fifty-two patients with metastatic solid tumors were enrolled in the Duke Cancer Center clinics over a ˜11 month time period. Patients were enrolled for each of the following: prostate adenocarcinoma (10 pts), renal cell carcinoma (RCC, 10 pts), colorectal adenocarcinoma (10 pts), urothelial carcinoma (8 pts), gastro-esophageal adenocarcinoma (7 pts), and pancreatic adenocarcinoma (7 pts). Efforts were made to enroll patients with resistant disease to VEGF (RCC) or EGFR (colon) inhibitors, or for men with bone metastatic CRPC (prostate) in order to enrich for c-MET expression. All patients had metastatic disease with predominantly lymph node, liver, lung, and bone metastases (Tables 2-A and 2-B). Most of the patients had undergone multiple lines of targeted and systemic chemotherapies. All of the prostate patients had received either combined androgen blockade or surgical castration, 9 had received either abiraterone or enzalutamide, 9 had received docetaxel, and all had bone metastases. Nine of the RCC patients (7 clear cell, 2 papillary and 1 collecting duct) were refractory to VEGF-targeting therapies including sunitinib, axitinib, and pazopanib. All 8 of the bladder cancer patients were refractory to previous platinum-based chemotherapy. Of the 7 gastro-esophageal cancer patients who were enrolled, 6 had received prior 5-FU//leucovorin (LV)/oxaliplatin, and 2 had received prior trastuzumab for HER2-positive disease. All of the colorectal cancer patients had been treated with 5-FU/LV/oxaliplatin, 9 had been treated with bevacizumab, and 6 had received prior EGFR targeting therapy (either cetuximab or panitumumab). Of the 7 pancreatic cancer patients, 6 had received prior 5-FU, 5 had received prior gemcitabine, 4 had received prior oxaliplatin, and 2 had received prior nab-paclitaxel. Further details are provided in Tables 2-A and 2-B. Tables 2-A and 2-B show baseline characteristics for all patients who underwent peripheral blood sampling. All continuous variables (hemoglobin, albumin, lactate dehydrogenase, and tumor markers) are summarized as median (range).

TABLE 2-A

| Disease | | Age | | | |
|---|---|---|---|---|---|
| site | | mean | | | Sites of metastases |
| (Total N) | Ethnicity | (range) | Sex | Prior therapies (N) | (N) |
| Prostate | 7 W, | 72 | 10 M | GnRH agonist (9), bicalutamide | LNs (5), |
| (10) | 3 AA | (53-83) | | (9), | Liver (5), |
| | | | | surgical castration (1), | Lung (2), |
| | | | | abiraterone or enzalutamide (9), | Bone (10) |
| | | | | sipuleucel-T (5), docetaxel (9), | |
| | | | | cabazitaxel (4) | |
| Renal cell | 10 W | 61 | 8 M | VEGF-targeted therapy (9), | LNs (6), |
| (10) | | (35-69) | | IL-2 (1), Gemcitabine/cisplatin (1) | Liver (4), |
| | | | | | Lung (8), |
| | | | | | CNS (2), |
| | | | | | Bone (6) |
| Bladder | 7 W, | 66 | 6 M | Gemcitabine/cisplatin (6), | LNs (7), |
| (8) | 1 AA | (56-77) | | carboplatin (2), radiation (3) | Liver (1), |
| | | | | | Lung (5), |
| | | | | | Bone (5) |
| Gastric | 5 W, | 70 | 4 M | 5-FU/oxaliplatin (6), | LNs (4), |
| (7) | 2 AA | (50-82) | | Trastuzumab (2), carboplatin- | Liver (4), |
| | | | | paclitaxel (1) | Lung (2), |
| | | | | | Bone (1), Ovaries (1) |
| Colon | 10 W | 55 | 5 M | EGFR-therapy (6), 5-FU (10), | LNs (9), |
| (10) | | (46-68) | | oxaliplatin (10), bevacizumab (9), | Liver (7), |
| | | | | irinotecan (9), regorafenib (5), | Lung (9), |
| | | | | aflibercept (3), mitomycin C (2), | CNS (1), |
| | | | | radiation (2) | Bone (1), Peritoneum |
| | | | | | (4) |
| Pancreas | 5 W, | 66 | 3 M | 5-FU (6), | LNs (3), |
| (7) | 2 AA | (54-73) | | oxaliplatin (4), | Liver (3), |
| | | | | gemcitabine (5), nab-paclitaxel (2), | Lung (6), Pleura (2), |
| | | | | irinotecan (1), ruxolitinib (1) | Peritoneum (1) |

TABLE 2-B

| | | Albumin | | |
|---|---|---|---|---|
| | Hemoglobin | (g/dL) | LDH (IU/L) | Tumor marker* |
| Disease site | (g/dL) | Median | Median | Median |
| (Total N) | Median (range) | (range) | (range) | (range) |
| Prostate (10) | 11.4 | 3.8 | 215 | 172 |
| | (8.3, 13.8) | (2.7, 4.7) | (158, 2812) | (9, 466) |
| Renal cell | 11.8 | 3.4 | 173 | |
| (10) | (8.8, 14) | (2.9, 4.3) | (103, 1131) | |
| Bladder (8) | 11.5 | 3.6 | 129 | |
| | (9.7, 12.3) | (3.3, 4.4) | (102, 738) | |
| Gastric (7) | 10 | 3.1 | n/a | |
| | (8.7, 13.6) | (1.3, 4.1) | | |
| Colon (10) | 12.1 | 3.5 | 221 | 23 |
| | (8.9, 13.4) | (2.8, 4.4) | (169, 668) | (1.5, 1433) |
| Pancreas (7) | 12.7 | 3.2 | n/a | 63 |
| | (8.1, 15.7) | (2.6, 4.3) | | (6, 608663) |

*Tumor markers: PSA in prostate cancer, CEA in colon cancer, and CA 19-9 in pancreatic cancer. Units are ng/mL for PSA and CEA; units/mL for CA 19-9.

Abbreviations: VEGF: vascular endothelial growth factor; IL-2: interleukin-2; EGFR: epidermal growth factor receptor; 5-FU: 5-fluorouracil; LDH: lactate dehydrogenase; W: White; AA: African American; M: male; CEA: carcinoembryonic antigen; CA 19-9: cancer antigen 19-9

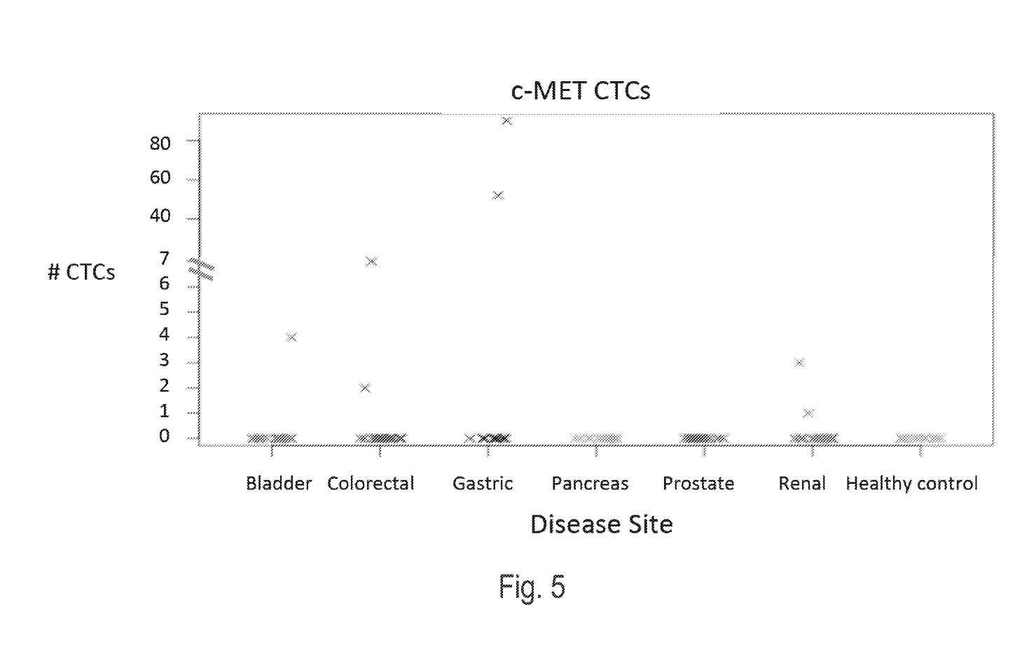

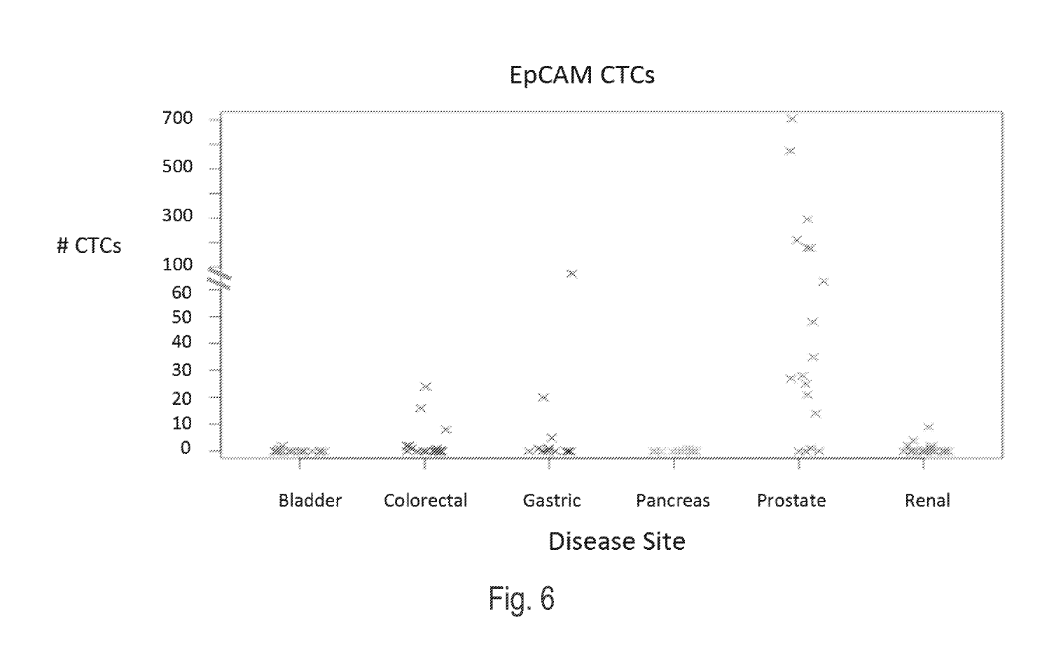

c-MET CTCs and EpCAM CTCs were enumerated in duplicate in all patients and summarized (Table 3). c-MET CTCs meeting the criteria described above (FIG. 3) were found in 4 patients (8%) (FIG. 4), and at least one EpCAM CTC was identified in 23 patients (44%) (FIG. 5). Of the 4 cases that had detectable c-MET CTCs, 3 cases were validated in replicate samples with MET amplification and trisomy 7 confirmed by DNA FISH, for a total cross-sectional prevalence of 6% (95% CI 1-16%). The subgroup cross-section point-estimated prevalence was 14% for gastric cancer, 10% for colorectal and RCC, and 13% for urothelial carcinoma. These cases are further described below in order to provide greater clinical context for the successful c-MET CTC capture events in this study. Table 2 shows cell types captured per disease site. N: number; CTCs: circulating tumor cells; EpCAM: epithelial cell adhesion molecule; CK: cytokeratin.

TABLE 3

| | | | | | | | | | | | EpCAM | |
|---|---|---|---|---|---|---|---|---|---|---|---|---|
| | | | Subjects | | c-MET capture | | | | Subjects | | capture | |
| | c-MET | | (N, %) with | | CD45+/CK+ | | EpCAM | | (N, %) with | | CD45+/CK+ | |
| | CTCs | | greater | | cells | | CTCs | | greater than | | cells | |
| Disease | (median, | | than 0 c- | | (median, | | (median, | | 0 EpCAM | | (median, | |
| site | range) | | MET CTCs | | range) | | range) | | CTCs | | range) | |
| Prostate | 0 | (0, 0) | 0 | (0%) | 0 | (1, 59) | 28 | (0, 705) | 9 | (90%) | 0 | (0, 2) |
| N = 10 | | | | | | | | | | | | |
| Renal cell | 0 | (0, 3) | 1 | (10%) | 2 | (0, 16) | 0 | (0, 9) | 3 | (30%) | 3 | (N/A*) |
| N = 10 | | | | | | | | | | | | |
| Bladder | 0 | (0, 4) | 1 | (13%) | 0 | (0, 15) | 0 | (0, 2) | 2 | (25%) | 12 | (1, 54) |
| N = 8 | | | | | | | | | | | | |
| Gastric | 0 | (0, 90) | 1 | (14%) | 4 | (0, 24) | 0 | (0, 20) | 3 | (43%) | 0.5 | (0, 13) |
| N = 7 | | | | | | | | | | | | |
| Colon | 0 | (0, 7) | 1 | (10%) | 0 | (0, 28) | 0 | (0, 24) | 5 | (50%) | 0 | (0, 0) |
| N = 10 | | | | | | | | | | | | |
| Pancreas | 0 | (0, 0) | 0 | (0%) | 1 | (0, 488) | 0 | (0, 1) | 1 | (14%) | 0 | (0, 0) |
| N = 7 | | | | | | | | | | | | |
| Overall | 0 | (0, 90) | 4 | (7.7%) | 1 | (0, 488) | 0 | (0, 705) | 23 | (44%) | 0.5 | (0, 54) |
| cancer | | | | | | | | | | | | |
| N = 52 | | | | | | | | | | | | |
| Healthy | 0 | (0, 0) | 0% | | 0 | (0, 11) | N/A | | N/A | | N/A | |
| control | | | | | | | | | | | | |
| N = 20 | | | | | | | | | | | | |

*EpCAM capture c-MET PE was performed for only 1 patient sample

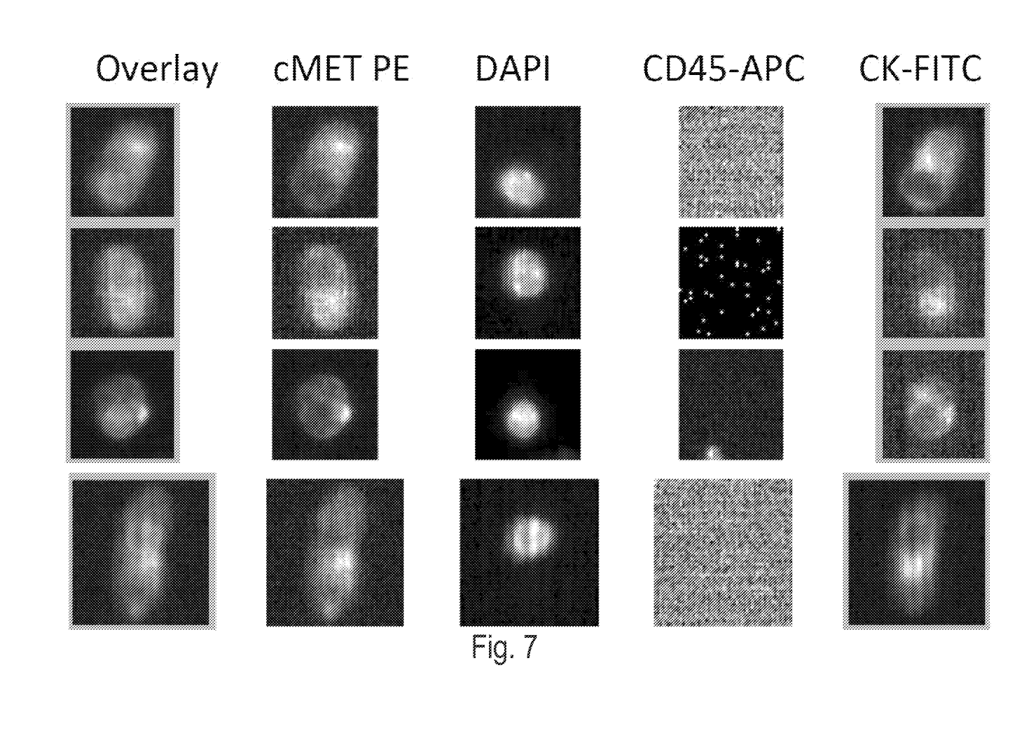

For each of FIGS. 7-10, the first column is combined fluorescent image of c-MET linked to PE and DAPI, the second column is c-MET linked to PE, the third column is DAPI staining, the fourth column is CD45 linked to APC, and the last column depicts cytokeratins linked to FITC.

Patient A was a 56-year-old Caucasian man with clear cell RCC, who had undergone nephrectomy but developed metastases in the liver, lungs, and pancreas. He had progression of disease after 8 months of pazopanib, was anemic, had an elevated LDH, and low albumin. Patient A was found to have 1 and 3 c-MET CTCs in duplicate samples (FIG. 7). He had 9 EpCAM CTCs in one of two samples (data not shown).

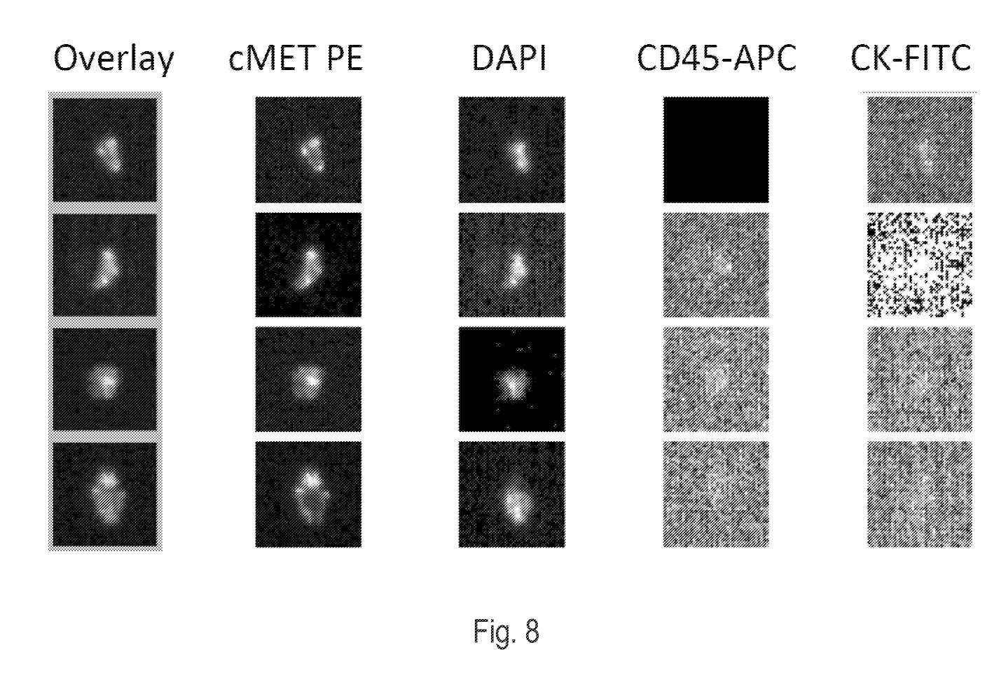

Patient B was a 65-year-old Caucasian man with metastatic urothelial carcinoma, with metastases in the left pelvis, and progression of disease in mediastinal lymph nodes and lung despite prior gemcitabine and cisplatin. Patient B was found to have 4 and 0 c-MET CTCs in duplicate samples (FIG. 8). Of note, patient B's cells were smaller in size and more elongated when compared to the other c-MET CTCs that were isolated. Patient B did not have any EpCAM CTCs in duplicate samples. As this sample did not replicate, these results were not includes the overall prevalence estimate as this finding could not be confirmed.

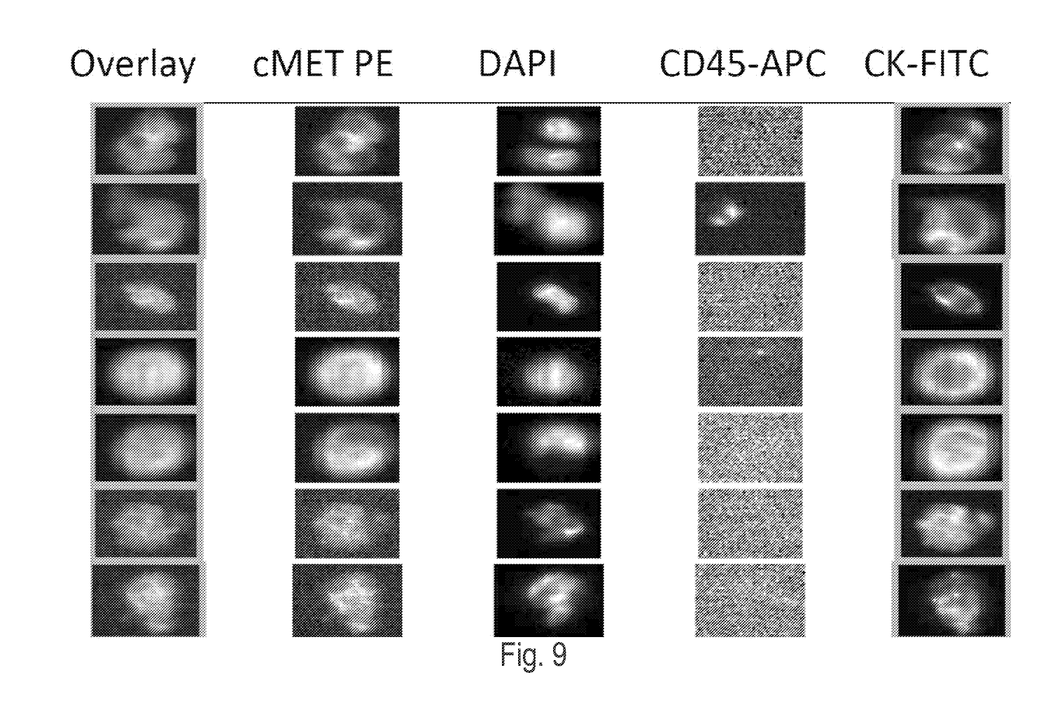

Patient C was a 64-year-old Caucasian man with adenocarcinoma at the gastroesophageal junction, with metastatic disease in lymph nodes, liver, and bone. His tumor was initially tested and found to be HER2 amplified, and he completed a course of therapy with 5-FU, LV, oxaliplatin, (FOLFOX) and trastuzumab. Peripheral blood samples were taken upon disease progression on this chemotherapy regimen. He was found to have 52 and 90 c-MET CTCs in duplicate samples (FIG. 9). He also had 20 and 69 EpCAM CTCs in duplicate samples. Once patient C was found to have a significant number of c-MET CTCs and MET amplification, he was treated with off-label crizotinib, a known c-MET tyrosine kinase inhibitor. He had rapid improvement of multiple areas of lymphadenopathy, with a 4-week clinical response, before experiencing disease progression and dying from metastatic disease.

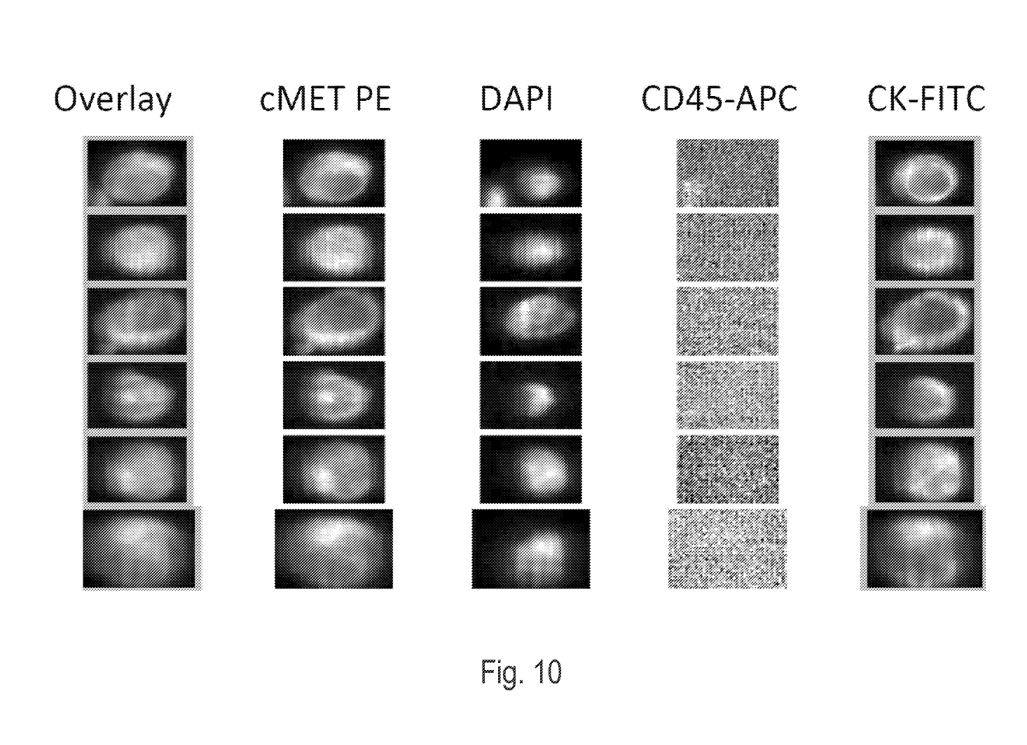

Patient D was a 55-year-old Caucasian woman with metastatic rectal cancer with regional recurrence in the presacral space, as well as disseminated metastases with retroperitoneal and bilateral hilar lymphadenopathy, hepatic lesions, and pulmonary nodules. She had undergone several lines of chemotherapy including FOLFOX with bevacizumab, FOLFIRI, panitumumab, cetuximab, regorafenib, as well as ziv-aflibercept. She was anemic, had an elevated LDH, low albumin and high CEA of 702.4 ng/mL. Patient D was found to have 7 and 2 c-MET CTCs in duplicate samples (FIG. 10).

The c-MET captured CTCs were then evaluated by FISH in order to determine the presence of any chromosome 7 gains or focal amplification of the c-MET locus, which would thus suggest a malignant origin to these CTCs. c-MET CTCs isolated from Patients A, C, and D underwent DNA FISH analysis for chromosome 7 and the MET gene. Patient A with clear cell renal cell carcinoma had trisomy 7 and three copies of the MET gene. Patient C with metastatic gastroesophageal adenocarcinoma had polysomy 7 and abundant MET gene amplification (MET/CEP7 ratio >10 in all tested CTCs). Patient D with metastatic colorectal cancer also had polysomy 7 and MET gene amplification (MET/CEP7 ratio >10 in all tested CTCs). Leukocytes in each sample underwent DNA FISH as internal control cells and had diploid chromosome 7 and two copies of the MET gene. These results suggest a malignant origin to the c-MET captured CTCs.

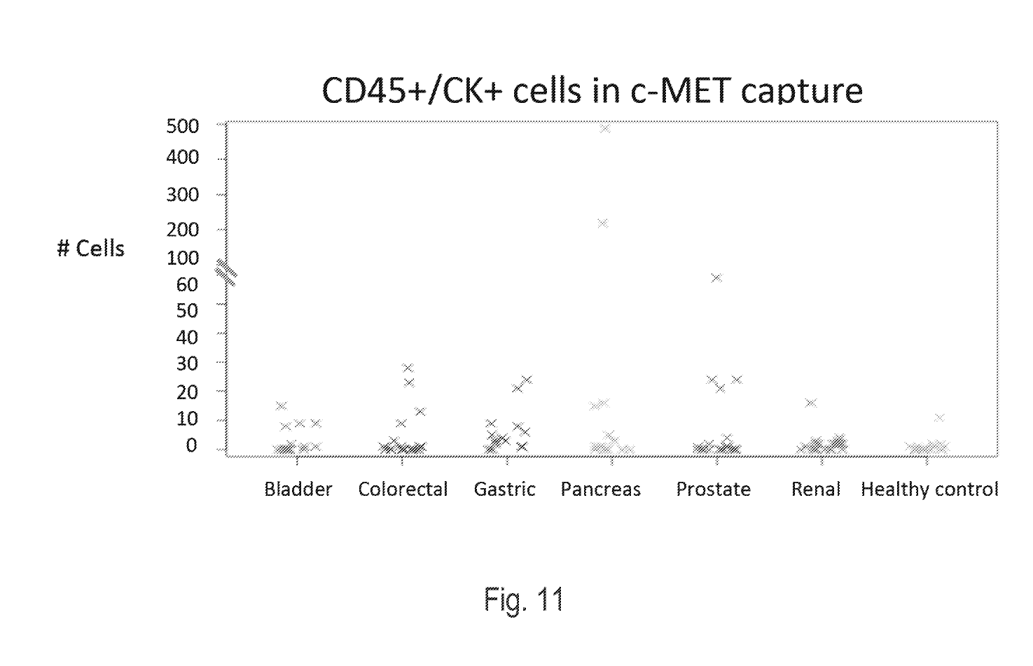

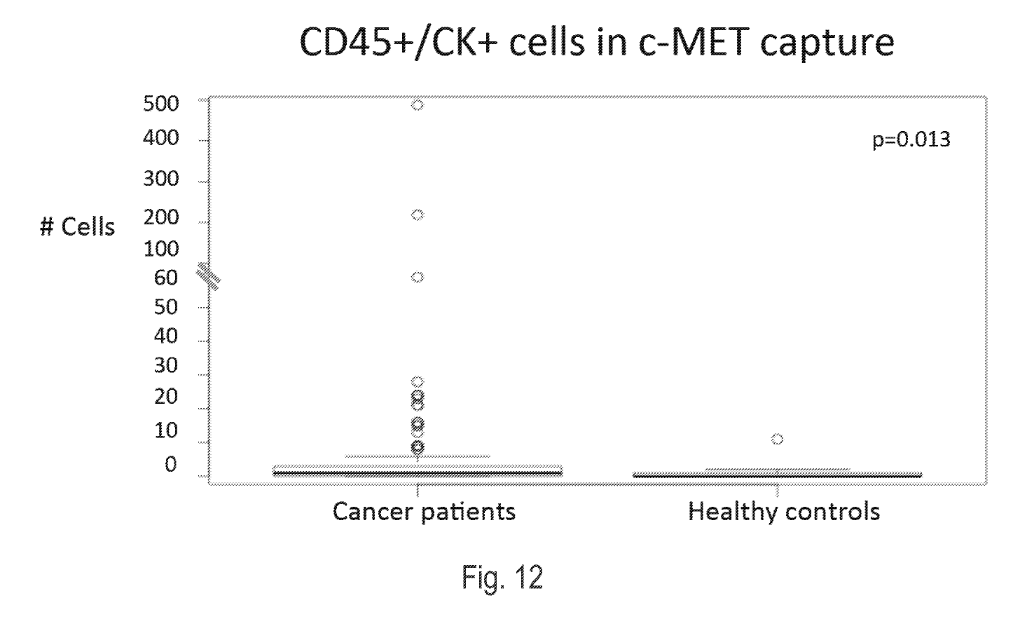

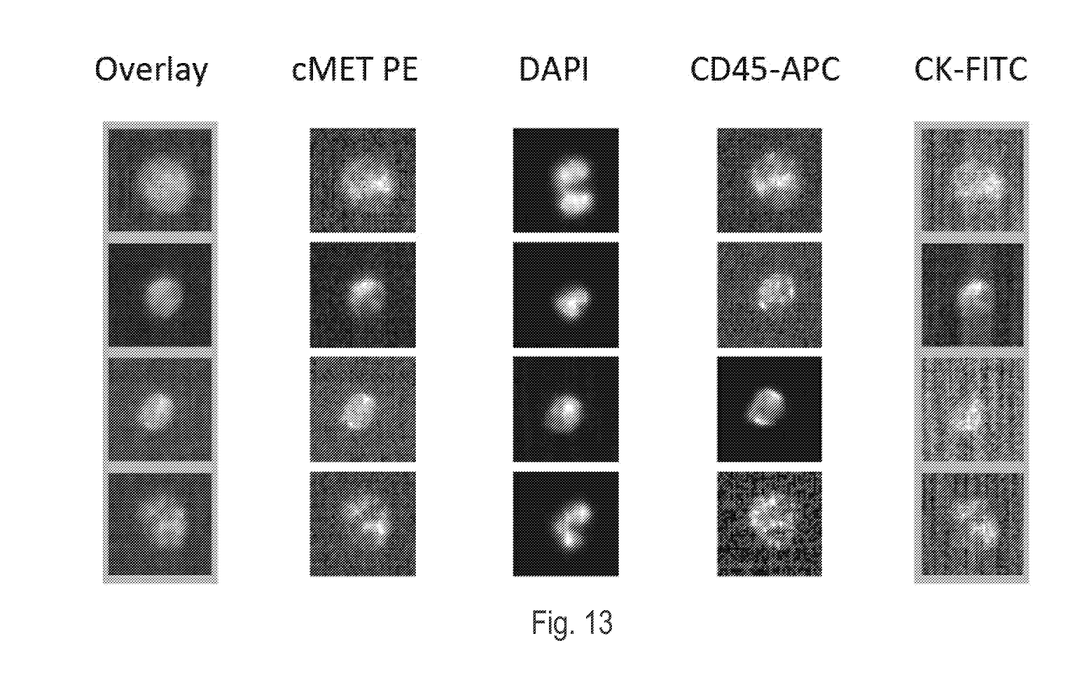

During isolation of c-MET CTCs, rare cells were also isolated and identified, which were positive for both CD45 and pan-cytokeratin, and which had a nuclear morphology consistent with benign cells. In 20 healthy control samples, 7 healthy controls had c-MET+/CD45+/CK+ cells, with 6 of 7 samples containing only 1 or 2 cells and only one healthy control with a 11 c-MET+/CD45+/CK+ cells (median 0 cells, prevalence 35%, 95% CI 14-56%). In 52 cancer patients enrolled on this study, 35 (67% prevalence, 95% CI 53-80%) had detectable c-MET+/CD45+/CK+ cells, with a median of 1 cell (range from 0 to 488). FIG. 11 is a plot of enumeration of the detectable c-MET+/CD45+/CK+ cells, separated by disease state. FIG. 12 is a boxplot of all samples of c-MET+/CD45+/CK+ cells from cancer patients versus healthy controls. Using a two-sided Wilcoxon rank sum test between cancer patients and healthy volunteers, the difference was statistically significant (p=0.013). These cells were smaller in morphology than c-MET CTCs described above, and some had bilobed nuclei (FIG. 13), suggestive of immature neutrophils. The number of c-MET+/CD45+/CK+ cells did not correlate with absolute neutrophil count (Spearman p-coefficient 0.10, p=0.49). When tested by DNA FISH, all of the c-MET+, CD45+ cells identified in cancer patients were diploid (data not shown), indicating their likely benign nature. However, the presence of these cells almost exclusively in cancer patients vs. healthy volunteers suggests that these c-MET circulating cells are highly associated with cancer.

This example has shown a novel, highly specific and minimally invasive assay for c-MET amplified CTCs based on c-MET capture and characterization of CTCs from the peripheral blood of multiple patients with metastatic carcinomas, including gastric, colorectal, and renal cell carcinomas. Importantly, cancer cells that over-express c-MET and lack EpCAM (like the HeLa and BT549 cell lines) can be captured with the c-MET CTC assay even when they are not detected by the Cellsearch® EpCAM assay, indicating loss of epithelial differentiation. The prevalence of a positive test (at least one detectable c-MET CTC) in our cohort was 6% to 14% (depending on disease site), similar to the expected prevalence of MET amplification in patients with metastatic cancer, and was not present in normal healthy volunteers. The prevalence of c-MET positive CTCs ranged from 0% in men with metastatic castration resistant prostate cancer to 14% in patients with metastatic gastric cancer, indicating the importance of tumor lineage and context for this assay.

c-MET positive and MET amplified CTCs can be isolated and characterized from patients with various malignancies and therefore present a new potential biomarker for these patients, potentially enabling clinical studies that utilize MET amplification as a predictive biomarker. We have reproducibly isolated c-MET CTCs from patients with metastatic, treatment-refractory renal cell carcinoma, gastroesophageal, and colorectal adenocarcinomas. Patients were selected for high tumor burdens and treatment refractoriness to VEGF or EGFR/HER2 based therapies, where c-MET may play a role in mediating resistance. Surprisingly, c-MET CTCs were not detected in the majority of patients, indicating that this assay may not detect c-MET overexpressing, non-amplified CTCs. This may be either due to cleavage of the c-MET extracellular domain (i.e. shedding), altered conformation of the c-MET extracellular domain, or relatively low abundance of c-MET expression on the cell surface of CTCs in the absence of gene amplification.

Of note, in three out of four cases, the presence of c-MET CTCs was linked to the presence of genomic changes in MET, with either trisomy 7 or MET amplification. Therefore, MET amplification and the degree of c-MET expression on the cell surface may be critical to the ability to capture these cells. Further studies are ongoing in selected patients with known MET amplification, or enriched for the context by which c-MET is commonly overexpressed, such as in non-small cell lung cancer or in patients harboring known tumor-specific genomic amplifications in the MET locus.

A high prevalence of CD45+ leukocytes co-expressing both cytokeratin and c-MET were found in patients with metastatic solid tumors but rarely in healthy volunteers. These cells were diploid and had the appearance of band neutrophils, an immature and activated myeloid cell type. Previous work has suggested the expression of activated c-MET signaling in phagocytic immune cells, which may represent the population of leukocytes that we have found. Recent work by Finisguerra et al. has also demonstrated the importance of c-MET on neutrophils for chemoattraction and cytotoxic killing of cancer cells. Therefore, it is theorized that this population of c-MET positive leukocytes is a biomarker of immune activation in the setting of cancer. Further studies of the prevalence of this c-MET-based leukocyte assay in early, localized cancer are needed to evaluate its clinical relevance.

In conclusion, a novel method for the isolation and characterization of c-MET expressing cells (CTCs and leukocytes) in patients with a diverse range of metastatic solid tumors has been developed, using a non-invasive and reproducible assay with high sensitivity and specificity. While the prevalence of a positive test in the observed cohort was low, this prevalence was consistent with the known prevalence of MET amplification in these patients, which is very rare in prostate cancer and relatively more common in treatment-refractory gastric and colorectal cancer. Given the association of c-MET CTCs with MET amplification, the presence of c-MET CTCs may be useful as a predictive biomarker for c-MET directed therapies.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosure described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of isolating a c-MET circulating tumor cell (CTC) from a patient, the method comprising:
a) obtaining a biological sample from the patient, the biological sample comprising the c-MET CTC;
b) contacting the biological sample or a fraction of the biological sample with an unbound complex, the unbound complex comprising a capture binding species linked to a solid phase, the contacting being for a time sufficient to allow the unbound complex to bind an extracellular binding domain of a c-MET protein on the c-MET CTC to form a bound complex, the capture binding species specifically binding the extracellular binding domain of the c-MET protein; and
c) isolating the bound complex.

2. A method of isolating an intact c-MET cell from a patient, the method comprising:
a) obtaining a biological sample from the patient, the biological sample comprising the intact c-MET cell;
b) contacting the biological sample or a fraction of the biological sample with an unbound complex, the unbound complex comprising a capture binding species linked to a solid phase, the contact being for a time sufficient to allow the unbound complex to bind an extracellular binding domain of a c-MET protein on the intact c-MET cell to form a bound complex, the capture binding species specifically binding the extracellular binding domain of the c-MET protein; and
c) isolating the bound complex.

3. The method of claim 1, the method further comprising removing at least a portion of the biological sample that does not include the c-MET CTC.

4. The method of claim 2, the method further comprising removing at least a portion of the biological sample that does not include the intact c-MET cell.

5. The method of claim 3 or 4, wherein the removing step comprises aspirating.

6. The method of any of the preceding claims, wherein step c) comprises aspirating unbound cells.

7. The method of any of the preceding claims, wherein step c) comprises applying an external magnetic field to the bound complex.

8. The method of claim 1, the method further comprising enumerating c-MET CTCs in the biological sample.

9. The method of claim 2, the method further comprising enumerating intact c-MET cells in the biological sample.

10. The method of any of the preceding claims, the method further comprising contacting the bound complex with a staining solution comprising a staining complex, the staining complex comprising a detectable label linked to a staining binding species.

11. The method of claim 10, wherein the staining binding species is an intracellular binding domain of the c-MET protein staining binding species that specifically binds an intracellular binding domain of the c-MET protein.

12. The method of claim 11, wherein the intracellular binding domain of the c-MET protein staining binding species comprises an intracellular binding domain of the c-MET protein binding portion that specifically binds to a protein having a polypeptide sequence of residues 956 to 1390 of SEQ ID NO: 1.

13. The method of claim 11 or 12, wherein the intracellular binding domain of the c-MET protein comprises a polypeptide sequence comprising at least a portion of residues 956 to 1390 of SEQ ID NO: 1.

14. The method of claim 10, wherein the staining binding species is a CD45 staining binding species that specifically binds CD45.

15. The method of claim 14, wherein the CD45 staining binding species comprises a CD45 binding portion that specifically binds a protein having a polypeptide sequence of SEQ ID NO: 2 or SEQ ID NO: 2 with a deletion of residues 32 to 192.

16. The method of claim 14 or 15, wherein CD45 comprises a polypeptide sequence comprising at least a portion of SEQ ID NO: 2.

17. The method of claim 10, wherein the staining binding species is a cytokeratin staining binding species that specifically binds a cytokeratin.

18. The method of claim 17, wherein the cytokeratin staining binding species comprises a cytokeratin binding portion that specifically binds a protein having a polypeptide sequence selected from the group consisting of SEQ ID NO: 3, SEQ ID NO: 4, and SEQ ID NO: 5.

19. The method of claim 17 or 18, wherein the cytokeratin comprises a polypeptide sequence selected from the group consisting of SEQ ID NO: 3, SEQ ID NO: 4, and SEQ ID NO: 5.

20. The method of any of claims 10 to 19, wherein the staining solution comprises 4′,6-diamidino-2-phynylindole (DAPI).

21. The method of any of claims 10 to 20, the method further comprising spectroscopically interrogating the detectable label.

22. The method of claim 20 or 21, the method further comprising spectroscopically interrogating DAPI.

23. The method of any of the preceding claims, wherein the extracellular binding domain of the c-MET protein comprises a polypeptide sequence comprising at least a portion of residues 25 to 932 of SEQ ID NO: 1.

24. The method of any of the preceding claims, wherein the capture binding species is a capture binding protein comprising an extracellular binding domain of the c-MET protein binding portion that specifically binds a protein comprising a polypeptide sequence comprising at least a portion of residues 25 to 932 of SEQ ID NO: 1.

25. The method of any of the preceding claims, wherein the patient has cancer.

26. The method of claim 25, wherein the cancer is gastric cancer, pancreatic cancer, renal cancer, colorectal cancer, bladder cancer, or prostate cancer.

27. The method of claim 25, wherein the cancer is gastric cancer, colorectal cancer, or renal cell carcinoma.

28. A method of isolating a c-MET circulating tumor cell (CTC) from a patient, the method comprising:
a) obtaining a blood sample from the patient, the blood sample comprising a cellular component and a non-cellular component;
b) optionally removing some or all of the non-cellular component from the blood sample;
c) contacting the cellular component with a ferrofluid comprising an unbound complex, the unbound complex comprising a capture binding protein linked to a magnetic particle, the contacting being for a time sufficient to allow the unbound complex to bind an extracellular binding domain of a c-MET protein on the c-MET CTC to form a bound complex, the capture binding species specifically binding the extracellular domain of the c-MET protein;
d) isolating the bound complex from unbound cells of the cellular component;
e) contacting the bound complex with a staining solution; and
f) spectroscopically interrogating the bound complex.

29. A ferrofluid comprising a ferromagnetic particle linked to a binding species that selectively binds to at least a portion of an extracellular domain of c-MET.


SEQUENCE LISTING (5 sequences)

SEQ ID NO: 1 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 2 - protein, 1304 aa, Homo sapiens
MYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 3 - protein, 483 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 4 - protein, 430 aa, Homo sapiens
MSFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 5 - protein, 400 aa, Homo sapiens
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL